(19) United States
(12) Patent Application Publication
(10) Pub. No.: US 2016/0317843 A9
(43) Pub. Date: Nov. 3, 2016

(54) INTEGRATED ABLATION AND MAPPING SYSTEM

(71) Applicants: James W. Arenson, Woodside, CA (US); Thilaka Sumanaweera, San Jose, CA (US); John P. Madden, Emerald Hills, CA (US)

(72) Inventors: James W. Arenson, Woodside, CA (US); Thilaka Sumanaweera, San Jose, CA (US); John P. Madden, Emerald Hills, CA (US)

(73) Assignee: VytronUS, Inc., Sunnyvale, CA (US)

(21) Appl. No.: 13/446,552

(22) Filed: Apr. 13, 2012

Related U.S. Application Data
(63) Continuation-in-part of application No. 13/405,712, filed on Feb. 27, 2012, now Pat. No. 8,511,317.
Application No. 13/405,712 is a continuation of application No. 13/092,747, filed on Apr. 22, 2011, now Pat. No. 8,146,603.
Application No. 13/092,747 is a continuation of application No. 11/747,862, filed on May 11, 2007, now Pat. No. 7,950,397.
(60) Provisional application No. 61/475,130, filed on Apr. 13, 2011.
(60) Provisional application No. 60/747,137, filed on May 12, 2006.
(60) Provisional application No. 60/919,831, filed on Mar. 23, 2007.

(51) Int. Cl. A61N 7/02

(52) U.S. Cl. CPC A61N 7/022

(57) ABSTRACT

A system for ablating and mapping tissue comprises a stand alone tissue ablation system adapted to ablate the tissue, and a stand alone cardiac mapping system adapted to map the tissue. The ablation system is operably coupled with the cardiac mapping system such that mapping data from the cardiac mapping system is provided to the ablation system to create a graphical display of the tissue and the ablation system position relative to the tissue. Motion of the ablation system may be monitored and adjusted based on feedback provided by ablation system actuators as well as position sensors.

### CROSS-REFERENCE

The present application is a non-provisional of, and claims the benefit of U.S. Provisional Patent Application No. 61/475,130 (Attorney Docket No. 31760-721.101) filed Apr. 13, 2011; the entire contents of which are incorporated herein by reference.

### BACKGROUND OF THE INVENTION

Atrial fibrillation (AF) is characterized by the abnormal and uncoordinated contraction of the atria and often the presence of an irregular ventricular response. In normal sinus rhythm, the electrical impulses originate in the sino-atrial node (SA node) which resides in the right atrium. The abnormal beating of the atrial heart muscle is known as fibrillation and is caused, in some cases, by electrical impulses originating in the pulmonary veins (PV) as reported by M. Haissaguerre et al., in “Spontaneous Initiation of Atrial Fibrillation by Ectopic Beats Originating in the Pulmonary Veins,” published in the New England J Med., Vol. 339:659-666.

There are pharmacological treatments for this condition with varying degrees of success. In addition, there are surgical interventions that are aimed at controlling the aberrant electrical signals in the left atrium (LA), such as the Cox-Maze III Procedure which has been described by J. L. Cox et al. in “The development of the Maze procedure for the treatment of atrial fibrillation,” published in Seminars in Thoracic & Cardiovascular Surgery, 2000; 12: 2-14. Other related publications include J. L. Cox et al., “Electrophysiologic basis, surgical development, and clinical results of the maze procedure for atrial flutter and atrial fibrillation,” published in Advances in Cardiac Surgery, 1995; 6: 1-67; and J. L. Cox et al., “Modification of the maze procedure for atrial flutter and atrial fibrillation. II, Surgical technique of the maze III procedure,” published in the Journal of Thoracic & Cardiovascular Surgery, 1995; 2110:485-95.

There has been considerable effort in developing catheter based systems for the treatment of AF to ablate some of the tissue that is the trigger for AF or to electrically isolate it. One such technique uses radiofrequency (RF) energy. Such methods are described in U.S. Pat. No. 6,064,902 to Haissaguerre et al.; U.S. Pat. No. 6,814,733 to Schwartz et al.; U.S. Pat. No. 6,996,908 to Maguire et al.; U.S. Pat. No. 6,955,173 to Lesh; and U.S. Pat. No. 6,949,097 to Stewart et al. Another such technique uses microwave energy. Such methods are described in U.S. Pat. No. 4,641,649 to Walinsky; U.S. Pat. No. 5,246,438 to Langberg; U.S. Pat. No. 5,405,346 to Grundy, et al.; and U.S. Pat. No. 5,314,466 to Stern, et al.; and U.S. Patent Publication Nos. 2002/0087151; 2003/0050631; and 2003/0050630 to Mody et al.

Another catheter based method utilizes a cryogenic technique where tissue of the atrium is frozen below a temperature of −60° C. Exemplary cryo-based devices are described in U.S. Pat. Nos. 6,929,639 and 6,666,858 to Lafontaine, and U.S. Pat. No. 6,161,543 to Cox et al.

More recent approaches for the treatment of atrial fibrillation involve the use of ultrasound energy. The target tissue of the region surrounding the pulmonary vein is heated using ultrasound energy emitted by one or more ultrasound transducers. One such approach is described by Lesh et al. in U.S. Pat. No. 6,502,576. Yet another catheter device using ultrasound energy is described by Gentry et al. in “Integrated Catheter for 3-D Intracardiac Echocardiography and Ultrasound Ablation,” published in the IEEE Transactions on Ultrasonics, Ferroelectrics, and Frequency Control, Vol. 51, No. 7, pp 799-807. Other devices based on ultrasound energy to create circumferential lesions are described in U.S. Pat. Nos. 6,997,925; 6,966,908; 6,964,660; 6,954,977; 6,953,460; 6,652,515; 6,547,788; and 6,514,249 to Maguire et al.; U.S. Pat. Nos. 6,955,173; 6,052,576; 6,305,378; 6,164,283; and 6,012,457 to Lesh; U.S. Pat. Nos. 6,872,205; 6,416,511; 6,254,599; 6,245,064; and 6,024,740; to Lesh et al.; U.S. Pat. Nos. 6,383,151; 6,117,101; and WO 99/02096 to Diederich et al.; U.S. Pat. No. 6,635,054 to Fjield et al.; U.S. Pat. No. 6,780,183 to Jimenez et al.; U.S. Pat. No. 6,605,084 to Acker et al.; U.S. Pat. No. 5,295,484 to Marcus et al.; and PCT Publication WO 2005/117734 to Wong et al.

While such ablation therapies alone are promising, it is preferred that ablation devices be used with guidance systems that indicate anatomical structures to aid in positioning the ultrasonic ablator with respect to the treatment region and guide the placement of the ablation energy. Current guidance capabilities rely on a variety of technologies, including X-ray fluoroscopy used alone or with ultrasound imaging, typically transesophageal or intracardiac echocardiography (ICE).

More recently, new types of cardiac mapping systems (CMS) are becoming more commonly used for providing guidance for catheter location in the atrium. These CMS create externally generated energy fields, usually electric fields or magnetic fields, which are detected via sensors in the distal end of the ablation catheters. The CMS can thereby locate the position of the tip of the catheter in 3-D space. Through a process of manipulating the tip of the catheter inside the atrium, the CMS collect a sequence of points adjacent to atrial walls and pulmonary veins, and use these data to render shapes representing the anatomical structure of the atrium. U.S. Pat. No. 5,738,096 to Ben-Haim discloses one such method for constructing a cardiac map.

Typically, these CMS rendered shapes of the atrium are obtained at the beginning of the ablation procedure. Subsequently, over a period of time as the ablations are created, the CMS sense the position of the distal end of the ablation catheter, as described in U.S. Pat. No. 6,690,963 to Ben-Haim et al., and superimpose the catheter position in these previously rendered anatomical shapes. A trail of dots or other graphic symbols are left on the rendered anatomical shapes corresponding to locations where a stand-alone RF generator drives the catheter so that its distal tip emits RF energy. Two commonly used CMS are the EnSite System from St. Jude Medical, as described in U.S. Pat. No. 7263,397 to Hauck et al., and the Carto 3 System from Biosense Webster, a Johnson & Johnson company, disclosed in U.S. Pat. No. 6,788,967.

These CMS also provide a means to collect and display intracardiac electrograms (IEGMs), a record of changes in electrical potentials detected from electrodes placed within the heart. The CMS superimpose color coded IEGM information indicating where the depolarizations originate in the heart, and their propagation patterns through the heart. IEGMs provide a useful adjunct to evaluating the progress and acute success of the AF ablation procedure.

An ablation system that includes an integrated cardiac mapping system is described in U.S. patent application Ser. No. 12/909,642 which includes a robotically controlled low intensity collimated ultrasound (LICU) catheter for treating AF. The low intensity collimated ultrasound energy beam provided by the catheter is described in more detail in U.S. Patent Publication No. 2007/0265609. The entire contents of both patent applications is incorporated herein by reference.

This LICU ablation system uses low intensity collimated ultrasound to form lesions through the use of an ultrasound beam, with sufficient energy to create lesions where the beam meets the tissue. Guiding formation of lesions is a map derived using ultrasound echoes from the collimated beam returning from endocardial structures.

The LICU ablation system is comprised of a catheter, a control console, remote control pod, and a robot pod that manipulates the catheter. The following describes a typical use of the system. The catheter is manually manipulated and deployed during introduction into the body and initial placement into the heart. Once in the distal end of the catheter is in the desired anatomic location and connected via the robot pod, the catheter tip responds to physician inputs at the control console or remote control pod. The catheter tip moves along a scanning pattern and a software algorithm in the control console processes A-mode ultrasound information to create estimates of the distance between the catheter tip and the endocardium, also referred to as gap values, at corresponding positions along the scan pattern. This gap information is rendered by system software and presented as a map on the display such that anatomical features and/or contours of the cardiac wall relative to the position of the catheter tip can be visualized.

The user then selects an appropriate target lesion trajectory, superimposed on the gap raster display. Finally, the physician selects the appropriate power and instructs the system to create the lesions along the specified trajectory in the cardiac wall. If desired, the physician may select different power levels and/or speed for different sections of the trajectory, and the system will adjust the output power accordingly as the beam moves along those sections of the trajectory. While lesions are being formed, the system provides real-time continuous monitoring of gap information and compares it to the previously acquired scan sweep information, and alerts the operator when patient movement may have occurred.

This LICU system provides contemporaneous guidance by ultrasonic means to locate the catheter tip in the heart, as well as a means to create consistent lesions of any shape and pattern. As physicians have become familiar and reliant on CMS information, it would be useful to combine LICU with CMS solutions. Furthermore, integrated IEGM information would provide useful adjunctive information to the clinicians using the LICU system.

In addition, the integrated CMS position information assists the LICU system to precisely control the position of the distal end of the catheter. When used alone, the LICU system manipulates and bends the tip of the catheter through actuators and sensors in or near the proximal end of the catheter. The LICU controller moves those actuators according to mathematical (algorithmic) models predicting the distal bending in response to the proximal actuators. These models of the mechanical transfer function may be imperfect, and can result in distal bending that deviates from the intended motion, even with feedback provided from proximal sensors. This distal end distortion would be greatly reduced if the LICU system could sense both the proximal positions of the actuators, and also the distal location of the catheter tip. The CMS system provides a means to unobtrusively sense the position of the distal end of the catheter. This CMS provided position data can be used to adjust and modify the actions of the proximal actuators, and thereby correct for any distortion introduced along the catheter. In an engineering sense, the position data from the CMS system is used to provide dynamic feedback in the closed loop catheter control system implemented in the LICU system.

The ablation system and the mapping systems are typically separate systems. It would be particularly useful to provide the guidance and ablation capabilities in a single unit. Furthermore, in a moving target such as the heart tissue, the original target, as identified by CMS, could move and non-target tissue could be ablated. Hence, contemporaneous (or almost contemporaneous) guidance and ablation will minimize the risk of ablating non-target tissue. Such guidance would assist the system or the operator to position the ablator with respect to the treatment region, to evaluate the treatment progression and to ensure that only the targeted tissue region is ablated. At least some of these objectives will be met by the embodiments disclosed herein.

### BRIEF SUMMARY OF THE INVENTION

The present application discloses a number of methods that combine Cardiac Mapping Systems (CMS) with Low Intensity Collimated Ultrasound (LICU) ablation systems. The resulting integration provides physicians with a more complete solution that provides catheter navigation, electrophysiology information, lesion formation and lesion verification in one system. Exemplary embodiments illustrate integration of guidance and therapy to create ablation zones in human tissue. More specifically, this disclosure pertains to the design of systems and methods for improving the treatment of atrial fibrillation of the heart using ultrasound energy, and more particularly to a medical device used for creating tissue lesions in specific locations in the heart.

In a first aspect of the present invention, a system for ablating and mapping tissue comprises a stand alone tissue ablation system adapted to ablate the tissue, and a stand alone cardiac mapping system adapted to map the tissue. The ablation system is operably coupled with the cardiac mapping system such that mapping data from the cardiac mapping system is provided to the ablation system to create a graphical display of the tissue and the ablation system position relative to the tissue.

In another aspect of the present invention, a system for ablating and mapping tissue comprises a stand alone tissue ablation system adapted to ablate the tissue, and a stand alone cardiac mapping system adapted to map the tissue. The ablation system is operably coupled with the cardiac mapping system such that data characterizing the tissue from the tissue ablation system is provided to the cardiac mapping system to create a graphical display of the tissue and the ablation system position relative to the tissue.

The tissue ablation system may comprise an actuatable catheter based ultrasound ablation system such as a low intensity collimated ultrasound ablation system. The catheter may comprise at least one sensing element adjacent a distal portion of the catheter. The at least one sensing element may be operably coupled with the cardiac mapping system.

The cardiac mapping system may be adapted to determine location of the at least one sensing element in space. The cardiac mapping system may graphically display the location of the at least one sensor superimposed on a representation of the tissue in a display device. The one or more sensors may be adapted to capture intracardiac electrogram signals from the tissue, and the intracardiac electrogram signals may be graphically displayed by either the cardiac mapping system or the ablation system. The cardiac mapping system may provide a video signal to the tissue ablation system, or the ablation system may provide a video signal to the cardiac mapping system. The video signal may be graphically displayed in a picture-in-picture display of a graphical display in the ablation system, or in the cardiac mapping system. The video signal may be graphically displayed in a separate monitor from an ablation system monitor. The separate monitor may display information from the cardiac mapping system. The video signal may be graphically displayed in a separate monitor from a cardiac mapping system monitor. The separate monitor may display information from the ablation system. Three dimensional data from the cardiac mapping system may indicate the positions of the sensors and this data may be provided to the ablation system and combined with three dimensional ablation system data. Three dimensional tissue data from the ablation system may be provided to the cardiac mapping system and combined with three dimensional mapping data. The combined three dimensional data may be graphically presented in a display. The cardiac mapping system data and the ablation system data may be scaled and aligned with one another.

In another aspect of the present invention, an integrated system for ablating and mapping tissue comprises a tissue ablation system adapted to ablate the tissue, and a cardiac mapping system adapted to map the tissue. The ablation system is integrated with the cardiac mapping system to form a single integrated system. The ablation system is operably coupled with the cardiac mapping system such that mapping data from the cardiac mapping system is provided to the ablation system to create a graphical display of the tissue and the ablation system position relative to the tissue.

In still another aspect of the present invention, an integrated system for ablating and mapping tissue comprises a tissue ablation system adapted to ablate the tissue, and a cardiac mapping system adapted to map the tissue. The ablation system is operably coupled with the cardiac mapping system such that data characterizing the tissue from tissue ablation system is provided to the cardiac mapping system to create a graphical display of the tissue and the ablation system position relative to the tissue.

In yet another aspect of the present invention, a system for ablating and mapping tissue comprises a stand alone tissue ablation system adapted to ablate the tissue, and a cardiac mapping system adapted to map the tissue. The ablation system is operably coupled with the cardiac mapping system such that mapping and guidance data from the cardiac mapping system is combined with ablation therapy data from the ablation system, the combined data graphically displayed by the system.

The tissue ablation system and the cardiac mapping systems may each be stand alone systems or they may be integrated into a single system.

In another aspect of the present invention, a method for ablating and mapping tissue comprises providing a tissue ablation system and providing a cardiac mapping system. Mapping the tissue is conducted with the cardiac mapping system, and data about the mapped tissue is captured. Tissue is ablated with the ablation system, and data about the ablated tissue captured. Tissue ablation data from the ablation system is provided to the cardiac mapping system, or cardiac mapping data from the cardiac mapping system is provided to the tissue ablation system. The tissue ablation data is combined with the cardiac mapping data. The combined data is then displayed on a monitor.

Mapping the tissue may comprise mapping position of the ablation system relative to the tissue. Mapping the tissue may comprise mapping a surface of the tissue. Ablating the tissue may comprise ultrasonically ablating the tissue with a low intensity collimated ultrasound beam. Combining the tissue ablation data with the cardiac mapping data may comprise scaling and aligning both data sets.

In another aspect of the present invention, a method for accurately bending and positioning the tip of the catheter comprises a cardiac mapping system providing position data to a tissue ablation system. The position data is used to provide feedback for the robotically controlled catheter to reduce distortion in the intended patterns of distal tip motion.

In still another aspect of the present invention, a method for ablating tissue comprises providing a tissue ablation system which comprises an ablation catheter, providing a cardiac mapping system, and sensing a field generated by a field generator with sensors on the ablation catheter thereby determining a position of a working end of the ablation catheter. The method also comprises actuating actuators operably coupled to the ablation catheter thereby moving the working end of the ablation catheter toward a target treatment site, detecting operating parameters associated with the position of the actuators, and providing the operating parameters to a control system associated with the tissue ablation catheter so as to provide feedback on tissue ablation catheter working end position. The method also comprises adjusting one or more of the actuators based on the feedback thereby positioning the working end of the catheter to a desired location appropriately near the target treatment site, providing output from the sensors to the cardiac mapping system and determining a second estimate of the position of the working end of the ablation catheter. The second estimate of position to the tissue ablation system is provided, and then the working end of the catheter is re-adjusted that the working end is properly located relative to the target treatment site.

The method may further comprise ablating tissue with the tissue ablation catheter. The ablation catheter may comprise an ultrasound ablation catheter. Detecting the operating parameters may comprise measuring one of force, displacement, rotation, and torque of one or more of the actuators. The sensors may be disposed on a distal portion of the ablation catheter and the actuators may be disposed adjacent a proximal portion of the ablation catheter.

In another aspect of the present invention, a method for ablating tissue comprises providing a tissue ablation system that has an ablation catheter, providing a cardiac mapping system, and measuring an electric potential from, or an impedance with an external power source using sensors on the ablation catheter thereby determining a first estimate of a position of a working end of the ablation catheter. The method also includes actuating actuators operably coupled to the ablation catheter thereby moving the working end of the ablation catheter toward a target treatment site, and detecting operating parameters associated with the position of the actuators. The operating parameters are provided to a control system associated with the tissue ablation catheter so as to provide feedback on tissue ablation catheter working end position. Adjusting one or more of the actuators based on the feedback positions the working end of the catheter appropriately to the target treatment site. Output from the sensors is then provided to the cardiac mapping system so that a second estimate of the position of the working end of the ablation catheter may be determined. The second estimate of position is provided to the tissue ablation system, and the position of the working end of the catheter is re-adjusted based on the second estimate so that the working end is closer to the target treatment site.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic view of a stand-alone CMS linked with a stand-alone LICU ablation system where the integrated information is displayed in the LICU system.

FIG. 2 is a schematic view of a stand-alone CMS linked with a stand-alone LICU ablation system where the integrated information is displayed in the CMS.

FIG. 3 a schematic view of a CMS integrated within a LICU ablation system.

FIG. 4 is a schematic view of a LICU ablation system integrated within a CMS.

FIG. 5 is a block diagram of a CMS system proving dynamic catheter tip position data to a LICU ablation system.

### DETAILED DESCRIPTION OF THE INVENTION

The following exemplary embodiments illustrate a medical system for guiding ablation of body tissue that combines the benefits of Cardiac Mapping Systems (CMS) with Low Intensity Collimated Ultrasound (LICU) ablation systems. Several configurations are included, each involving different approaches for mating the CMS and LICU systems, so that data can be shared thereby realizing the benefits of an integrated solution.

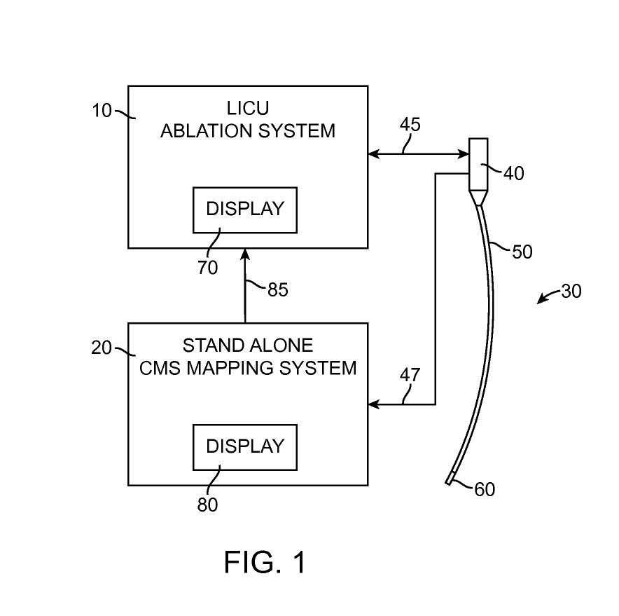

FIG. 1 shows diagrammatically a LICU Ablation System 10 linked to a stand-alone Cardiac Mapping System (CMS) 20. Catheter 30, comprised of a catheter handle 40, a catheter body 50 and distal end 60, is operably connected to and controlled by LICU 10 as indicated by arrow 45, which provides both mechanical means to manipulate the catheter, as well as electrical means to drive and sense ultrasound from the distal end 60. One or more sensing elements such as electrodes (not illustrated) located in the catheter distal end 60 are operably connected to the CMS 20 as indicated by arrow 47. CMS 20 determines the location of the sensing leads in the distal end 60 in space, using normal CMS techniques, and displays that position superimposed on a graphic representation of the atrium on the CMS display 80. CMS can also derive IEMG signals and display those potentials detected from the electrodes in distal end 60.

There are a number of different methods to integrate the CMS 20 derived information into the LICU system 10. One approach is to send a video signal, as indicated by arrow 85 containing the information shown on CMS display 80 to LICU 10, which in turn displays this video signal in a PIP (picture-in-picture) area of the LICU display 70. Those persons reasonably skilled in the art of video processing are familiar with techniques for displaying one video image over an area of a second video image. A simplified approach is to provide a second display monitor as part of the LICU system 10, and dedicate this monitor exclusively to display CMS supplied information.

Alternatively, CMS 20 provides a 3-D data set that includes the derived X, Y, Z positions of the sensors in distal end 60 located in three space (X,Y,Z) inside the heart. This 3-D data is sent to LICU 10, where it is combined with LICU 3-D data and presented on display 70.

To make use of a single integrated display of the two sets of 3-D data, the two sets of data need to be scaled and aligned. In one approach the LICU system 10 moves the catheter distal end 60 to multiple (at least three) distinct locations in three space as reference points. At each reference point the LICU system 10 queries the CMS 20 to provide detected 3-D locations. These reference data points provide sufficient information for the LICU system 10 to scale and align complete CMS 3-D data sets with the LICU 3-D data sets. Then the two 3-D data sets can be combined and presented on display 70. Those reasonably skilled in the art can provide alternative methods for scaling and aligning two sets of 3-D data.

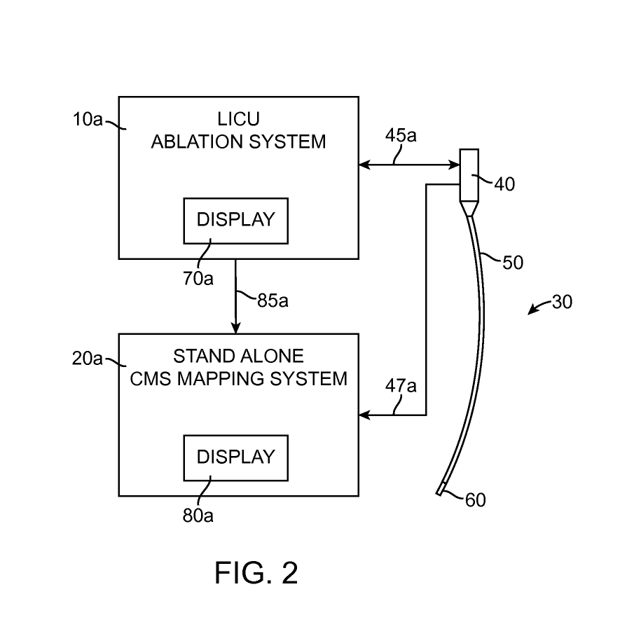

FIG. 2 shows diagrammatically a stand-alone Cardiac Mapping System (CMS) 20a linked to a LICU Ablation System 10a. Catheter 30, comprised of a catheter handle 40, a catheter body 50 and distal end 60, is operably coupled with and controlled by LICU 10a, as indicated by arrow 45a, which provides both mechanical means to manipulate the catheter, as well as electrical means to drive and sense ultrasound from the distal end 60. One or more sensing elements such as electrodes (not illustrated) located in the catheter distal end 60 are operably connected to the CMS 20a as shown by arrow 47a. CMS 20a determines the location of the sensing leads in the distal end 60 in space, using normal CMS techniques, and displays that position superimposed on a graphic representation of the atrium on the CMS display 80a. CMS can also derive IEMG signals and display those potentials detected from the leads in distal end 60.

There are a number of different methods to integrate the LICU system 10a derived information into CMS 20a, as illustrated with arrow 85a. One approach is to send a video signal containing the information shown on LICU display 70a to CMS 20a, which in turn displays this video signal in a PIP (picture-in-picture) area of the CMS display 80a. Those persons reasonably skilled in the art of video processing are familiar with techniques for displaying one video image over an area of a second video image. A simplified approach is to provide a second display monitor as part of CMS 20a, and dedicate this monitor exclusively to display LICU supplied information.

Alternatively, LICU system 10a provides a 3-D data set that includes the X, Y, Z locations corresponding to the LICU displayed information. This 3-D data is sent to CMS 20a, where it is combined with CMS 3-D data and presented on display 80a.

To make use of a single integrated display of the two sets of 3-D data, the two sets of data need to be scaled and aligned. In one approach the LICU system 10a moves the catheter distal end 60 to multiple (at least three) distinct locations in three space as reference points. At each reference point the LICU system 1a captures that 3-D location and informs the CMS 20a to likewise capture the corresponding 3-D location. These reference data points provide sufficient information for the CMS 2a to scale and align complete LICU 3-D data sets with the CMS 3-D data sets. Then the two 3-D data sets can be combined and presented on display 80a. Those reasonably skilled in the art can provide alternative methods for scaling and aligning two sets of 3-D data.

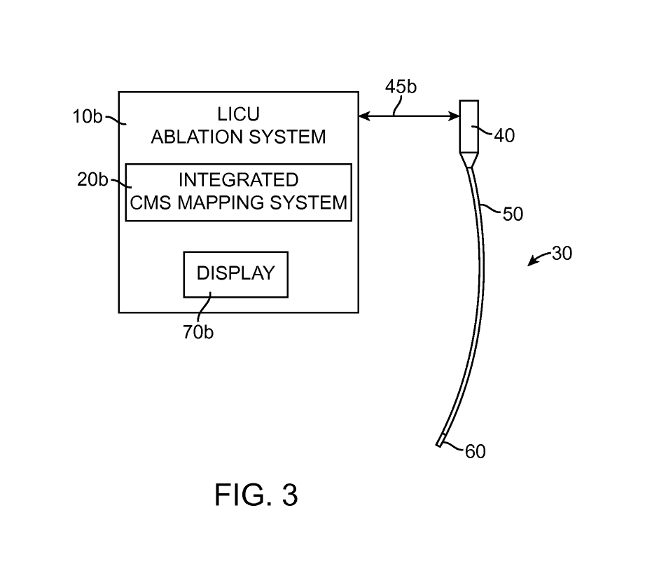

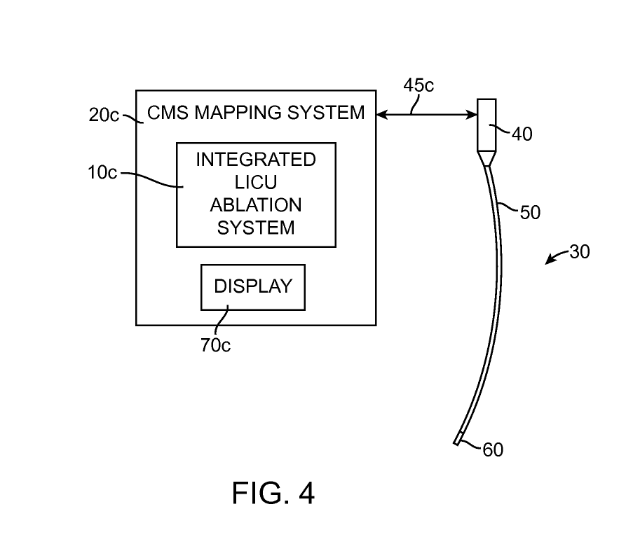

FIG. 3 diagrammatically shows LICU system 10b with a completely integrated cardiac mapping system (ICMS) 20b operatively coupled with catheter 30 as illustrated by arrow 45b. The ICMS 20b is integrated hardware and software derived from stand-alone CMS 20 or 20a. Alternatively, the features may be implemented directly in the LICU system 10c (see FIG. 4) by modifying existing LICU system hardware and software. Alternatively, a hybrid of both ICMS and LICU system hardware and software may be used. Alternatively, the fully integrated LICU system 10 or 10a may use modules provided by third party (OEM) vendors such as Ascension Technology Corporation (Milton, Vt.) which provide 3-D tracking devices. These modules are specifically designed to integrate into existing medical systems. This integrated solution has an advantage over those shown in FIG. 1 and FIG. 2 in that they take up less space in the operating room, and can be controlled by a single operator.

FIG. 4 diagrammatically shows CMS 20c with a completely integrated LICU ablation system 10c that is operatively coupled to catheter 30 as indicated by arrow 45c. The Integrated LICU system 10c may be integrated hardware and software derived from stand-alone LICU system 10, or 10a, or the features may be implemented directly in the CMS system 20c by modifying existing hardware and software, or a combination of both. Alternatively, the fully integrated CMS 20c may use modules provided by third party (OEM) vendors that provide functionality comparable to a LICU ablation system. This fully integrated solution has an advantage over those shown in FIG. 1 and FIG. 2 in that they take up less space in the operating room, and can by controlled by a single operator. A display 70c such as a video monitor graphically illustrates anatomic mapping, catheter position, and ablation information.

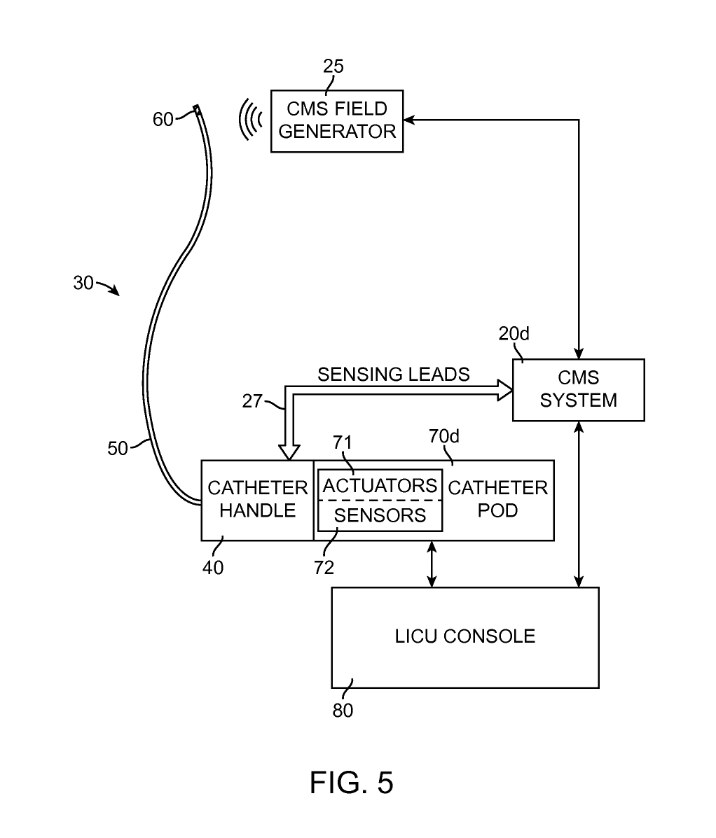

FIG. 5 shows an exemplary embodiment of the components to provide precision control of the distal end of the catheter. One of skill in the art will appreciate that other methods of determining and controlling position may also be used, such as by using impedance as will be discussed later. Catheter 30 is made up of a distal end 60, catheter body 50 and catheter handle 40. Distal end 60 includes sensors appropriate for detecting the field generated by CMS field generator 25. Catheter Handle 40 couples into the catheter pod 70d (also known as “robot”) which includes actuators 71 controlled from the LICU console 80. The actuators 71 impart forces on mechanical members in the catheter handle 40 which are ultimately translated into bending or steering motions of the distal end 60. Sensors 72 detect the forces or displacement or rotation or torque of the actuators, and provide feedback so that the actuators will move in a controlled fashion using feedback control systems familiar to those skilled in the art. The outputs of the sensors in the distal end 60 are connected to the CMS system 20d via a cable 27 from the catheter handle 40. The CMS system 20d calculates the position information of the distal end 60, and provides that data to the LICU console 80, where it is used as another feedback channel in the catheter tip position control system. Additional sensors in the catheter handle 40 may augment or replace the sensors 72 in the catheter pod 70d.

In an alternative embodiment to that described above in FIG. 5, instead of using a field generator, the system uses electrical potentials to determine position information of distal end 60 of catheter 30. This may be accomplished by placing pairs of cutaneous patches onto a patient, preferably three pairs of patches on three orthogonal axes. A low amplitude electrical signal is emitted from the patches and received by sensors such as electrodes on the distal end 60 of the catheter. Distal end 60 location is then determined by measuring the electrical potential or field strength by the catheter. This may also be accomplished by measuring or calculating the corresponding impedance. Other aspects of the system generally take the same form as previously described with respect to FIG. 5 above.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for ablating and mapping tissue, said system comprising:
a stand alone tissue ablation system adapted to ablate the tissue; and
a stand alone cardiac mapping system adapted to map the tissue,
wherein the ablation system is operably coupled with the cardiac mapping system such that mapping data from the cardiac mapping system is provided to the ablation system to create a graphical display of the tissue and the ablation system position relative to the tissue.

2. The system of claim 1, wherein the tissue ablation system comprises an actuatable catheter based ultrasound ablation system.

3. The system of claim 2, wherein the tissue ablation system comprises a low intensity collimated ultrasound ablation system.

4. The system of claim 2, wherein the catheter comprises at least one sensing element adjacent a distal portion of the catheter, the at least one sensing element operably coupled with the cardiac mapping system.

5. The system of claim 4, wherein the cardiac mapping system is adapted to determine location of the at least one sensing element in space, and wherein the cardiac mapping system graphically displays the location of the at least one sensing element superimposed on a representation of the tissue in a display device.

6. The system of claim 5, wherein the one or more sensors are adapted to capture intracardiac electrogram signals from the tissue, and wherein the intracardiac electrogram signals are graphically displayed by either the cardiac mapping system or the ablation system.

7. The system of claim 5, wherein the cardiac mapping system provides a video signal to the tissue ablation system.

8. The system of claim 7, wherein the video signal is graphically displayed in a picture-in-picture display of a graphical display in the ablation system.

9. The system of claim 7, wherein the video signal is graphically displayed in a separate monitor from an ablation system monitor, the separate monitor displaying information from the cardiac mapping system.

10. The system of claim 1, wherein the cardiac mapping system data and data from the ablation system are scaled and aligned with one another.

11. A system for ablating and mapping tissue, said system comprising:
a stand alone tissue ablation system adapted to ablate the tissue; and
a stand alone cardiac mapping system adapted to map the tissue,
wherein the ablation system is operably coupled with the cardiac mapping system such that data characterizing the tissue from the tissue ablation system is provided to the cardiac mapping system to create a graphical display of the tissue and the ablation system position relative to the tissue.

12. The system of claim 11, wherein the tissue ablation system comprises an actuatable catheter based ultrasound ablation system.

13. The system of claim 12, wherein the tissue ablation system comprises a low intensity collimated ultrasound ablation system.

14. The system of claim 12, wherein the catheter comprises at least one sensing element adjacent a distal portion of the catheter, the at least one sensing element operably coupled with the cardiac mapping system.

15. The system of claim 14, wherein the cardiac mapping system is adapted to determine location of the at least one sensing element in space, and wherein the cardiac mapping system graphically displays the location of the at least one sensing element superimposed on a representation of the tissue in a display device.

16. The system of claim 15, wherein the one or more sensors are adapted to capture intracardiac electrogram signals from the tissue, and wherein the intracardiac electrogram signals are graphically displayed by either the cardiac mapping system or the ablation system.

17. The system of claim 15, wherein the ablation system provides a video signal to the cardiac mapping system.

18. The system of claim 17, wherein the video signal is graphically displayed in a picture-in-picture display of a graphical display in the cardiac mapping system.

19. The system of claim 17, wherein the video signal is graphically displayed in a separate monitor from a cardiac mapping system monitor, the separate monitor displaying information from the ablation system.

20. The system of claim 15, wherein three dimensional tissue data from the ablation system is provided to the cardiac mapping system and combined with three dimensional mapping data, and the combined three dimensional data is graphically presented in a display.

21. The system of claim 20, wherein the cardiac mapping system data and the ablation system data are scaled and aligned with one another.

22. An integrated system for ablating and mapping tissue, said integrated system comprising:
a tissue ablation system adapted to ablate the tissue; and
a cardiac mapping system adapted to map the tissue,
wherein the ablation system is integrated with the cardiac mapping system to form a single integrated system, and wherein the ablation system is operably coupled with the cardiac mapping system such that mapping data from the cardiac mapping system is provided to the ablation system to create a graphical display of the tissue and the ablation system position relative to the tissue.

23. An integrated system for ablating and mapping tissue, said integrated system comprising:
a tissue ablation system adapted to ablate the tissue; and
a cardiac mapping system adapted to map the tissue,
wherein the ablation system is operably coupled with the cardiac mapping system such that data characterizing the tissue from tissue ablation system is provided to the cardiac mapping system to create a graphical display of the tissue and the ablation system position relative to the tissue.

24. A system for ablating and mapping tissue, said system comprising:
a stand alone tissue ablation system adapted to ablate the tissue; and
a cardiac mapping system adapted to map the tissue,
wherein the ablation system is operably coupled with the cardiac mapping system such that mapping and guidance data from the cardiac mapping system is combined with ablation therapy data from the ablation system, the combined data graphically displayed by the system.

25. The system of claim 24, wherein the tissue ablation system and the cardiac mapping systems are each stand alone systems.

26. The system of claim 24, wherein the tissue ablation system and the cardiac mapping systems are integrated into a single system.

27. A method for ablating and mapping tissue, said method comprising:
providing a tissue ablation system;
providing a cardiac mapping system;
mapping the tissue with the cardiac mapping system;
capturing data about the mapped tissue;
ablating the tissue with the ablation system;
capturing data about the ablated tissue; and
providing tissue ablation data from the ablation system to the cardiac mapping system, or providing cardiac mapping data from the cardiac mapping system to the tissue ablation system;
combining the tissue ablation data with the cardiac mapping data; and
graphically displaying the combined data on a monitor.

28. The method of claim 27, wherein mapping the tissue comprises mapping position of the ablation system relative to the tissue.

29. The method of claim 27, wherein mapping the tissue comprises mapping a surface of the tissue.

30. The method of claim 27, wherein ablating the tissue comprises ultrasonically ablating the tissue with a low intensity collimated ultrasound beam.

31. The method of claim 27, wherein combining the tissue ablation data with the cardiac mapping data comprises scaling and aligning both data sets.

32. A method for ablating tissue, said method comprising:
providing a tissue ablation system, the tissue ablation system comprising an ablation catheter;
providing a cardiac mapping system;
sensing a field generated by a field generator with sensors on the ablation catheter thereby determining a position of a working end of the ablation catheter;
actuating actuators operably coupled to the ablation catheter thereby moving the working end of the ablation catheter toward a target treatment site;
detecting operating parameters associated with the position of the actuators;
providing the operating parameters to a control system associated with the tissue ablation catheter so as to provide feedback on tissue ablation catheter working end position;
adjusting one or more of the actuators based on the feedback thereby positioning the working end of the catheter appropriately to the target treatment site;
providing output from the sensors to the cardiac mapping system and determining a second estimate of the position of the working end of the ablation catheter;
providing the second estimate of position to the tissue ablation system; and
re-adjusting the position of the working end based on the second estimate so that the working end is closer to the target treatment site.

33. The method of claim 32, further comprising ablating tissue with the tissue ablation catheter.

34. The method of claim 32, wherein the ablation catheter comprises an ultrasound ablation catheter.

35. The method of claim 32, wherein detecting the operating parameters comprise measuring one of force, displacement, rotation, and torque of one or more of the actuators.

36. The method of claim 32, wherein the sensors are disposed on a distal portion of the ablation catheter.

37. The method of claim 32, wherein the actuators are disposed adjacent a proximal portion of the ablation catheter.

38. A method for ablating tissue, said method comprising:
providing a tissue ablation system, the tissue ablation system comprising an ablation catheter;
providing a cardiac mapping system;
measuring an electric potential from, or an impedance with an external power source using sensors on the ablation catheter thereby determining a first estimate of a position of a working end of the ablation catheter;
actuating actuators operably coupled to the ablation catheter thereby moving the working end of the ablation catheter toward a target treatment site;
detecting operating parameters associated with the position of the actuators;
providing the operating parameters to a control system associated with the tissue ablation catheter so as to provide feedback on tissue ablation catheter working end position;
adjusting one or more of the actuators based on the feedback thereby positioning the working end of the catheter appropriately to the target treatment site;
providing output from the sensors to the cardiac mapping system and determining a second estimate of the position of the working end of the ablation catheter;
providing the second estimate of position to the tissue ablation system; and
re-adjusting the position of the working end based on the second estimate so that the working end is closer to the target treatment site.

39. The method of claim 38, further comprising ablating tissue with the tissue ablation catheter.

40. The method of claim 38, wherein the ablation catheter comprises an ultrasound ablation catheter.

41. The method of claim 38, wherein detecting the operating parameters comprise measuring one of force, displacement, rotation, and torque of one or more of the actuators.

42. The method of claim 38, wherein the sensors are disposed on a distal portion of the ablation catheter.

43. The method of claim 38, wherein the actuators are disposed adjacent a proximal portion of the ablation catheter.
